(19) United States
(12) Patent Application Publication
(10) Pub. No.: US 2017/0069188 A1
(43) Pub. Date: Mar. 9, 2017

(54) SYSTEM AND METHOD FOR REDUCING ALARM FATIGUE

(71) Applicant: COVIDIEN LP, Mansfield, MA (US)

(72) Inventors: Paul Stanley Addison, Edinburgh (GB); James N. Watson, Edinburgh (GB)

(21) Appl. No.: 14/848,661

(22) Filed: Sep. 9, 2015

(51) Int. Cl. G08B 19/00; A61B 5/00; G08B 26/00

(52) U.S. Cl. CPC G08B 19/00; G08B 26/008; A61B 5/7242; A61B 5/7282; A61B 5/746

(57) ABSTRACT

A method for reducing alarm fatigue, specifically, combining multiple physiological parameters, such as heart rate and respiratory rate, into one index number indicative of the patient's condition. The method includes detecting the severity of the patient's condition, generating, and displaying a scaled version of that index number relative to the severity of the patient's condition. The scaled index number is displayed on a patient monitor device. The scaled index number may have a size and color value.

### BACKGROUND

Hospitals utilize patient monitoring systems to keep track of different patient conditions such as blood pressure and respiratory rate. These patient monitoring systems may include alarms for when conditions meet a certain threshold. Present systems generate large quantities of audible and visual alarms. Exposure to an excessive number of alarms may lead to desensitization to the alarms and missed alarms.

### SUMMARY

The present disclosure relates to a method for reducing alarm fatigue, specifically, combining multiple physiological parameters, such as heart rate and respiratory rate, into one index number indicative of the patient's condition. The method includes detecting the severity of the patient's condition, generating, and displaying a scaled version of that index number relative to the severity of the patient's condition. The scaled index number is displayed on a patient monitor device. The scaled index number has a size and color value.

Certain embodiments of the present disclosure may provide one or more technical advantages. For example, certain technical advantages are described below.

Alarm fatigue is generally understood as a situation where clinicians are desensitized to alarms. Alarm fatigue can be prevalent especially where clinicians have prolonged exposure to many, frequent alarms. It can be appreciated that an alarm management system that reduces the total number of audible and visual alarms a clinician is exposed to could lessen this problem. In certain embodiments, this could mean integrating at least two physiological parameters into one index number indicative of a patient's condition.

Alarm fatigue can be prevalent in cases of alarm systems that fail to differentiate alarms of vital importance with alarms of lesser importance. It can be appreciated that visual alarms that are more attention grabbing could reduce this problem. In certain embodiments, this could mean increasing the size or changing the color of a displayed vital parameter relative to the severity of the condition it represents.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

### BRIEF DESCRIPTION OF THE DRAWINGS

For a more complete understanding of the present disclosure and its features and advantages, reference is now made to the following description, taken in conjunction with the accompanying drawings, in which:

FIG. 1 illustrates an example system for monitoring a patient according to certain embodiments of the present disclosure;

FIG. 2 illustrates an example system for controlling alarm fatigue according to certain embodiments of the present disclosure;

FIG. 3 illustrates an example system for controlling alarm fatigue according to certain embodiments of the present disclosure;

FIG. 4 illustrates an example system for controlling alarm fatigue according to certain embodiments of the present disclosure, and

FIG. 5 illustrates an example method for controlling alarm fatigue according to certain embodiments of the present disclosure.

### DESCRIPTION OF EXAMPLE EMBODIMENTS

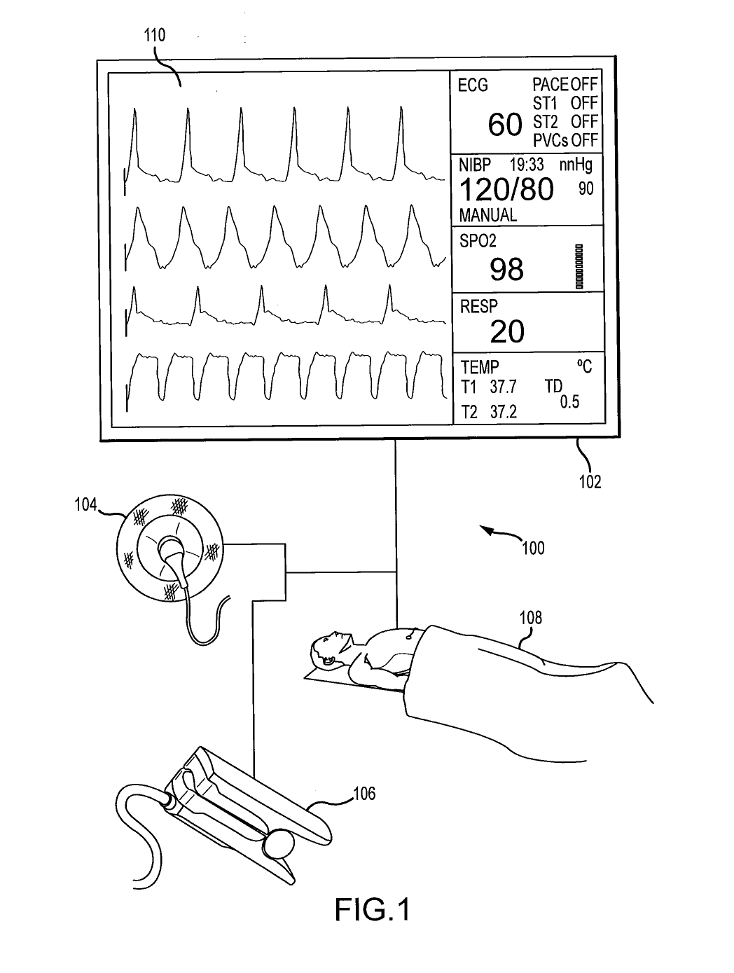

FIG. 1 illustrates an example system 100 for monitoring a patient and controlling alarm fatigue, according to certain embodiments of the present disclosure. System 100 includes one or more patient monitor devices 102, 104, 106, connected to one or more patients 108. In certain embodiments, system 100 is operable to collect certain physiological parameters, such as heart rate and respiratory rate, via patient monitor devices 102, 104, 106 from one or more patients 108 and present them on one or more patient monitor devices 102. In certain embodiments, patient monitor devices 102, 104, 106 may integrate two or more patient parameters into at least one index number. This index number along with other physiological parameters may be presented as data 110 on the display of one or more patient monitor devices 102.

Patient monitor device 102, for example, may act as, or be connected to, data collection servers that store physiological parameters collected from patient monitor devices 104, 106. In the example, patient monitor device 102 may include a mobile monitoring device. Patient monitor device 106 may include, for example, a pulse oximetry sensor. Patient monitor device 104 may include, for example, an electrocardiogram (ECG) sensor. Patient monitor device 102 may include one or more displays for presenting data 110 collected from patient monitor devices 104, 106. Patient monitor device 102 may be connected with remote devices for receiving and transmitting data 110. In other embodiments, patient monitor device 102 may include one or more sensors for reading physiological parameters directly from patients 108.

Physiological parameters may refer to any patient identifiers, medical history, clinician notes, alarm thresholds, alarm events, device settings, measurements of values indicating physiological conditions such as oxygen saturation levels, pulse rates, heart rates, other vital signs, and any other output data from monitor devices such as patient monitor devices 104, 106. Specifically, such parameters may include, but are not limited to: blood pressure, heart rate, respiratory rate, temperature, and consciousness.

In certain embodiments, patient monitor device 102 may be configured to integrate at least two physiological parameters to generate at least one index number indicative of a patient condition. For example, the index number may be formed from consecutive integers ranging from 1-10. The index number may include fractions or decimals such as “2½” or “3.7”. In certain embodiments, the index number may not be a number. In these example embodiments, the index number may be a range of colors that, for example, shift from green to yellow to red. In certain other embodiments, the index number may be a series of roman numerals, alphabetic letters, other symbols, or any combination of the above.

In certain embodiments, patient monitor device 102 may be configured to detect a severity of the patient condition based on the at least one index number. Relating to one of the above embodiments, the most severe patient condition may generate an index value of “1” in one example while the least severe patient condition may generate an index value of “10” and so on. The terms used to describe the severity of these patient conditions and corresponding index values may be any generic medical state such as “good, fair, stable, serious, critical, etc.” For example, an index value of “1” may relate to the most severe patient condition such as “critical” whereas an index value of “10” may relate to the least severe patient condition such as “good” or “stable.” The range of values need not be 1-10, they may be reversed (10-1), or they may be any conceivable appropriate set of values.

In certain embodiments, patient monitor device 102 may be configured to generate a scale factor for the at least one index number based on the detected severity. In one example embodiment, the index number indicating the most severe patient condition (such as a “1”) may have a scale factor of 10:1 whereas the index number indicating the least severe patient condition (such as a “10”) may have a scale factor of 1:1. It can be contemplated that groupings of index numbers (such as 1-3, 4-6, etc.) may have the same scale factor. For example, index numbers 1-3 may have a scale factor of 3:1, index numbers 4-6 may have a scale factor of 2:1, and so on. In certain embodiments, the rate of increase of the scale factor per the increase of severity of the patient condition can be linear, exponential, or any other conceivable function.

In certain embodiments, patient monitor device 102 may be configured to generate a scaled index number based on the generated scale factor, the scaled index number having a size and a color value. For example, generated scale factor may influence the displayed scaled index number to draw a clinician's attention to the scaled index number. In certain embodiments, the size of the scaled index number may be increased based on a generated scale factor, which may be an indication that the patient condition is critical. In certain embodiments, the color value of the scaled index number may be yellow or red to indicate that the patient condition is critical. For example, in these embodiments, the scale factor may be 3:1 for an index number of “2” and a critical patient condition. In this example, the display size for the index number “2” may be appropriately scaled on a screen to the scale factor 3:1 to alert a clinician to the condition. In this example, if the index number changes from “2” to “10” indicating the least severe patient condition, the scale factor may change to 1:1 and reduce the display of the index number to accommodate the display of other data 110.

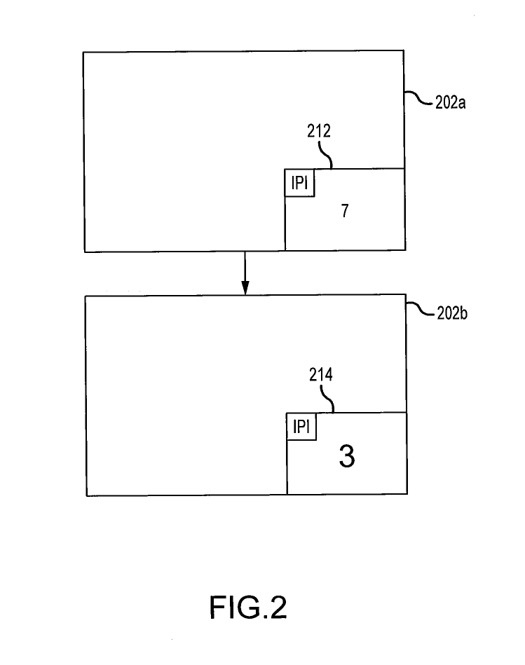

In certain embodiments, the scaled index number may indicate changes to patient condition to a clinician by other display features. As described above, low or dropping scaled index number may indicate a deteriorating patient condition. In the example, as the index number falls, indicating gradually more severe patient conditions, the scale factor may increase and, correspondingly, the size of the index number may grow relative to the area of the monitor display. In these example embodiments, the other displayed physiological parameters may shrink in displayed size in order to accommodate display of the scaled index number. In another embodiment, the base unit in the scale factor may not relate to a percentage of the monitor display, but relate to a standard size represented in fractions, standards of measure, exponential values, or other similar values. In another embodiment, in conjunction with a change in size or acting independently, the index number may be given a color value indicative of the severity of a patient condition. For example, the index number indicating the least severe patient condition may have a color scheme with the highest amount of green and the lowest amount of red. Conversely, the index number indicating the most severe patient condition may have a color scheme with the highest amount of red and the lowest amount of green. In still further embodiments, it can be contemplated that the index value may dynamically grow, shrink, change colors, flicker, flash, or any other conceivable movement to alert healthcare professionals, to show trends or patterns with changing index numbers, or for any other conceivable purpose. Additional details and examples of the influence of the generated scaled index number are described below with reference to FIGS. 2-5.

In certain embodiments, patient monitor device 102 may be configured to transmit the scaled index number to a display. For example, the transmitted scaled index number may be shown in conjunction with, or in displacement of, other data 110 previously shown on said display. It can be contemplated that said displays may include computer monitors, television screens, tablets, phones, laptops or any other conceivable patient monitors capable of receiving and displaying information. In certain embodiments, patient monitor device may transmit other data related to the scaled index number such as audio alerts, vibrations, or other data or communications to indicate an alarm or changes to patient status.

In certain embodiments, patient monitor device 102 may include a general purpose computer that includes a storage device, a scaled index number controller, a processor, a memory, a communication interface (I/F), an output device, and an input device, which are discussed in further detail below. The scaled index number controller may include any suitable logic embodied in non-transitory computer-readable media such as the storage device or memory, and when executed, that is operable to integrate at least two physiological parameters to generate at least one index number indicative of a patient condition. The at least two physiological parameters may be, for example a pulse oximetry value from patient monitor device 106 and an ECG value from patient monitor device 104. The scaled index number controller is further operable to detect a severity of the patient condition based on the at least one index number; generate a scale factor for the at least one index number based on the detected severity; generate a scaled index number based on the generated scale factor, the scaled index number having a size and a color value; and transmit the scaled index number to a display, such as the output device. Although this particular implementation of patient monitor device 102 is primarily described, the present disclosure contemplates any suitable implementation of patient monitor device 102 according to particular needs.

Although this particular implementation of system 100 is illustrated and primarily described, the present disclosure contemplates any suitable implementation of system 100 according to particular needs. Additional details of example embodiments of system 100 and patient monitor device 102 are discussed below with reference to FIGS. 2-5.

FIG. 2 illustrates an example system for controlling alarm fatigue according to certain embodiments of the present disclosure. FIG. 2 includes a patient monitor device 202a in a first state and a patient monitor device 202b in a second state. Patient monitor devices 202a and 202b may be substantially similar to patient monitor device 102 of FIG. 1. In FIG. 2, element 212 is a displayed index number. In the displayed example, the index number is an Integrated Pulmonary Index (IPI) value indicating a patient's respiratory state. The disclosure contemplates display of any index value. In the displayed example element 212 has a lesser severity (in this case, a “7”). Element 214 is a displayed index number of greater severity (in this case, a “3”). In this embodiment, as the index number decreases numerically from 7 to 3, the index number increases in size relative to the monitor to which it is displayed upon. This is accomplished in certain embodiments, by patient monitor device 202 generating a scale factor for the index number based on the detected severity and generating a scaled index number based on the generated scale factor as described above in FIG. 1.

In addition, as described above in FIG. 1, the displayed index number may be on any portion of the monitor of any size and may have any suitable display characteristics to indicate patient condition. For example, the displayed index number may have colors that change with the rise and fall of the severity of the patient's condition. As another example, the displayed index number may dynamically grow, shrink, change colors, flicker, flash, or any other conceivable movement to alert healthcare professionals, to show trends or patterns with changing index numbers, or for any other conceivable purpose.

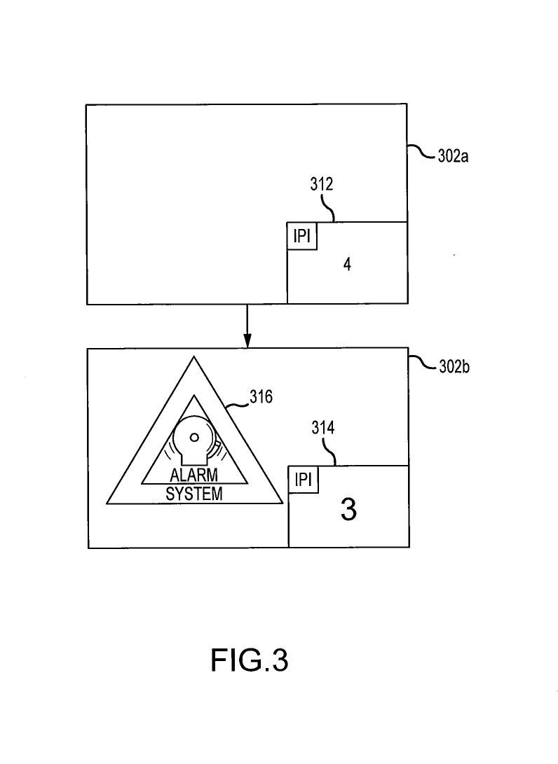

FIG. 3 illustrates an example system for controlling alarm fatigue according to certain embodiments of the present disclosure. FIG. 3 includes a patient monitor device 302a in a first state and a patient monitor device 302b in a second state. Patient monitor devices 302a and 302b may be substantially similar to patient monitor device 102 of FIG. 1. In FIG. 3, element 312 is a displayed index number of lesser severity (in this case, a “4”). Element 314 is a displayed index number of greater severity (in this case, a “3”). In this example system, element 316 is an alarm which signifies that the patient's condition has crossed a threshold and reached a critical status. The threshold may be a set automatic function of the software of the system or a practitioner may manually input the threshold. The threshold may be at any level of severity of the patient's condition the practitioner sets. Alarm 316 may include any suitable noise or level of vibration. Alarm 316 may be emitted locally from the apparatus or be transmitted wirelessly to other medical apparatuses, computers, tablets, phones, laptops or any other conceivable machine capable of receiving and displaying or emitting an alarm. Alarm 316 may be emitted locally and transmitted wirelessly. Alarm 316 may be triggered by an index number falling below a certain range or by any of the individual patient conditions used to calculate the index number falling below a certain range. In certain embodiments, display of alarm 316 may be scaled in relation to displayed index number 314 such that alarm 316 takes up an appropriate space on the display. All of the examples discussed in the description of FIGS. 1 and 2 may be incorporated into examples discussed for FIG. 3.

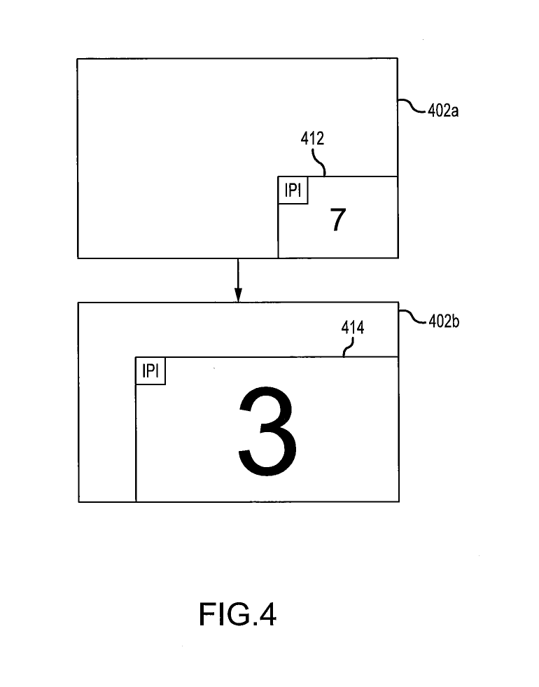

FIG. 4 illustrates an example system for controlling alarm fatigue according to certain embodiments of the present disclosure. FIG. 4 includes a patient monitor device 402a in a first state and a patient monitor device 402b in a second state. Patient monitor devices 402a and 402b may be substantially similar to patient monitor device 102 of FIG. 1. In FIG. 4, element 412 is a displayed index number of lesser severity (in this case, a “7”). Element 414 is a displayed index number of greater severity (in this case, a “3”). In this embodiment, as the index number falls from 7 to 3, the index number increases in size relative to the monitor to which it is displayed upon. As opposed to the example in FIG. 2, the embodiment of FIG. 4 may displace other information on the monitor as the index number increases in size. For example, with reference to FIG. 1, the data 110 on patient monitor device may be displayed on patient monitor device 402a. Such data may be reduced in display size in order to display critical information in element 414. All of the examples discussed in the description of FIGS. 1, 2, and 3 may be incorporated into examples discussed for FIG. 4.

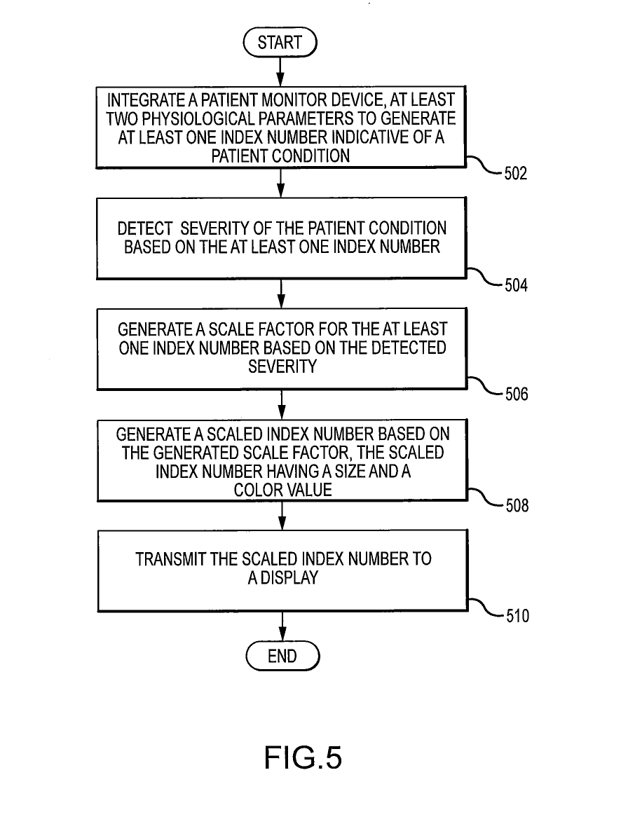

FIG. 5 illustrates an example method for controlling alarm fatigue according to certain embodiments of the present disclosure. The method begins at step 502 where a patient monitor device may integrate at least two physiological parameters to generate at least one index number indicative of a patient condition. For example, and as described above, the index number may be formed from consecutive integers ranging from 1-10. The index number may include fractions or decimals such as “2½” or “3.7” in other examples.

The method continues at step 504 where the patient monitor device may detect the severity of the patient condition based on the at least one index number. For example, the most severe patient condition may generate an index value of “1” in one example while the least severe patient condition may generate an index value of “10” and so on.

The method continues at step 506 where the patient monitor device may generate a scale factor for the at least one index number based on the detected severity. A scaled index number controller may generate the scale factor. For example, index numbers 1-3 may have a scale factor of 3:1, index numbers 4-6 may have a scale factor of 2:1, and so on. In certain embodiments, the rate of increase of the scale factor per the increase of severity of the patient condition can be linear, exponential, or any other conceivable function.

The method continues at step 508 where the patient monitor device may generate a scaled index number based on the generated scale factor with the scaled index number having a size and a color value. For example, generated scale factor may influence the displayed scaled index number to draw a clinician's attention to the scaled index number. In certain embodiments, the size of the scaled index number may be increased based on a generated scale factor, which may be an indication that the patient condition is critical. In certain embodiments, the color value of the scaled index number may be yellow or red to indicate that the patient condition is critical. For example, in these embodiments, the scale factor may be 3:1 for an index number of “2” and a critical patient condition. In this example, the display size for the index number “2” may be appropriately scaled on a screen to the scale factor 3:1 to alert a clinician to the condition. In this example, if the index number changes from “2” to “10” indicating the least severe patient condition, the scale factor may change to 1:1 and reduce the display of the index number to accommodate the display of other data 110.

In certain embodiments, the scaled index number may indicate changes to patient condition to a clinician by other display features. As described above, low or dropping scaled index number may indicate a deteriorating patient condition. In the example, as the index number falls, indicating gradually more severe patient conditions, the scale factor may increase and, correspondingly, the size of the index number may grow relative to the area of the monitor display. In these example embodiments, the other displayed physiological parameters may shrink in displayed size in order to accommodate display of the scaled index number. In another embodiment, the base unit in the scale factor may not relate to a percentage of the monitor display, but relate to a standard size represented in fractions, standards of measure, exponential values, or other similar values. In another embodiment, in conjunction with a change in size or acting independently, the index number may be given a color value indicative of the severity of a patient condition. For example, the index number indicating the least severe patient condition may have a color scheme with the highest amount of green and the lowest amount of red. Conversely, the index number indicating the most severe patient condition may have a color scheme with the highest amount of red and the lowest amount of green. In still further embodiments, it can be contemplated that the index value may dynamically grow, shrink, change colors, flicker, flash, or any other conceivable movement to alert healthcare professionals, to show trends or patterns with changing index numbers, or for any other conceivable purpose.

The method concludes at step 510 where the patient monitor device transmits the index number to a display. The transmitted scaled index number may be shown in conjunction with, or in displacement of, other information previously shown on said display. It can be contemplated that said displays may include computer monitors, television screens, tablets, phones, laptops or any other conceivable patient monitors capable of receiving and displaying information.

Although the present disclosure has been described with several embodiments, diverse changes, substitutions, variation, alteration, and modification may be suggested to one skilled in the art, and it is intended that the disclosure encompass all such changes, substitutions, variation, alterations, and modifications as fall within the spirit and scope of the appended claims.

1. A computer-implemented method for monitoring a patient, comprising:
integrating, at a patient monitor device, at least two physiological parameters to generate at least one index number indicative of a patient condition;
detecting a severity of the patient condition based on the at least one index number;
generating a scale factor for the at least one index number based on the detected severity;
generating a scaled index number based on the generated scale factor, the scaled index number having a size and a color value; and
transmitting the scaled index number to a display.

2. The method of claim 1, wherein the size of the scaled index number changes corresponding to the detected severity of the patient condition.

3. The method of claim 1, wherein the color value of the scaled index number changes corresponding to the detected severity of the patient condition.

4. The method of claim 1, further comprising triggering an alarm when the scaled index number passes a certain threshold.

5. The method of claim 1, wherein the displayed index number increases in size, while together displayed patient parameters decrease in size, as the detected severity of the patient condition increases.

6. A patient monitor device, comprising:
a processor;
a non-transitory memory coupled to the processor and storing instructions operable to:
integrate at least two physiological parameters to generate at least one index number indicative of a patient condition;
detect a severity of the patient condition based on the at least one index number;
generate a scale factor for the at least one index number based on the detected severity;
generate a scaled index number based on the generated scale factor, the scaled index number having a size and a color value; and
transmit the scaled index number to a display.

7. The device of claim 6, wherein the size of the scaled index number changes corresponding to the detected severity of the patient condition.

8. The device of claim 6, wherein the color value of the scaled index number changes corresponding to the detected severity of the patient condition.

9. The device of claim 6, wherein the instructions are further operable to trigger an alarm when the scaled index number passes a certain threshold.

10. The device of claim 6, wherein the displayed index number increases in size, while other displayed patient parameters decrease in size, as the detected severity of the patient condition increases.
